(19) United States
(12) Patent Application Publication
(10) Pub. No.: US 2022/0240753 A1
(43) Pub. Date: Aug. 4, 2022

(54) INSERTION INSTRUMENT

(71) Applicant: OLYMPUS CORPORATION, Tokyo (JP)

(72) Inventors: Natsuki HORI, Yokohama-shi (JP); Hidetsugu TANAKA, Hanno-shi (JP); Ryuhei FUJIMOTO, Tokyo (JP)

(73) Assignee: OLYMPUS CORPORATION, Tokyo (JP)

(21) Appl. No.: 17/724,610

(22) Filed: Apr. 20, 2022

Related U.S. Application Data
(63) Continuation of application No. PCT/JP2019/041992, filed on Oct. 25, 2019.

(51) Int. Cl. A61B 1/005

(52) U.S. Cl. CPC A61B 1/0052; A61B 2090/0807

(57) ABSTRACT

An insertion instrument includes: an insertion portion including a bending portion; an operation portion; one operation lever provided to the operation portion, the operation lever being configured to tilt, to cause the bending portion to bend in a predetermined direction; a movable member provided around the operation lever and configured to move according to tilting of the operation lever; and at least one indicator configured to change according to a movement of the movable member, the at least one indicator being visually recognizable.

### CROSS-REFERENCE TO RELATED APPLICATION

This application is a continuation application of PCT/JP2019/041992 filed on Oct. 25, 2019, the entire contents of which are incorporated herein by this reference.

### BACKGROUND OF THE INVENTION

### 1. Field of the Invention

The present invention relates to an insertion instrument, such as an endoscope, a treatment instrument, a medical manipulator, and the like, which includes one operation lever for causing a bending portion to bend in a predetermined direction.

### 2. Description of the Related Art

In recent years, endoscopes, which are insertion instruments, have been widely used in medical fields. Endoscopes for use in the medical fields are capable of observing a site to be examined in a body by inserting an elongated insertion portion into the body.

In addition, a configuration of an endoscope is known in which a bending portion configured to be bendable in a predetermined direction is provided on a distal end side of the endoscope. A flexible endoscope including a flexible insertion portion and a rigid endoscope including a rigid insertion portion are known.

A bending portion is configured to be bent to improve an advancing performance of an insertion portion at a flexed part of a lumen in a subject, and to change an observation direction of an observation optical system provided at a distal end portion located on a distal end side with respect to the bending portion.

Bending operation of the bending portion is performed by an operator operating a bending operation member. The bending operation member is provided to an operation portion provided continuously with a proximal end of the insertion portion of the endoscope.

A known example of the bending operation member includes an operation lever of a joystick apparatus configured to be tiltable from a neutral position at which the bending portion is in a non-bending state in a direction corresponding to each of various bending directions of the above-described bending portion.

In addition, known functions of such a joystick apparatus include a function for bringing a bending portion into a non-bending state and a hold function for holding a bending state of the bending portion. In the former function, if an operator tilts an operation lever and then takes the hand off from the operation lever, that is, releases the tilting operation, the operation lever returns to a neutral position to thereby bring the bending portion into the non-bending state. In the latter function, if the operator tilts the operation lever and then holds the tilting state of the operation lever, the bending state of the bending portion is held.

Note that the operator can bring the operation lever back to the neutral position even during the use of the hold function.

The operation lever is provided to an operation portion so as to stand up from one surface of an exterior member of the operation portion. The operation lever is constituted of a standing shaft and a finger contact member provided at an end of the shaft. The periphery of the standing shaft of the operation lever is covered with a watertight member which is an elastic member. Such a configuration prevents water from entering the operation portion.

Japanese Patent Application Laid-Open Publication No. H6-105799 discloses a configuration in which an operation portion is provided with an operation lever, and marks respectively indicating up, down, left, and right directions are provided around the operation lever.

With these marks, the operator can bring the operation lever back to a neutral position by causing the shaft of the operation lever to be positioned at the center of the marks, which are drawn respectively as U (up), D (down), L (left), and R (right) on the operation portion so as to surround the operation lever, that is, by causing the shaft of the operation lever to be positioned at a position equidistant from the respective marks.

In addition, when the operator takes the hand off from a finger contact member, if the shaft of the operation lever is positioned at the center of the marks, the operator can visually recognize that the operation lever is at the neutral position.

### SUMMARY OF THE INVENTION

An insertion instrument according to an aspect of the present invention includes: an insertion portion configured to be inserted into a subject, the insertion portion including a bending portion configured to be bendable in a predetermined direction; an operation portion provided on a proximal end side with respect to the insertion portion; one operation lever provided to the operation portion, the operation lever being configured to tilt, to cause the bending portion to bend in the predetermined direction; a movable member provided around the operation lever, and configured to move according to tilting of the operation lever; and at least one indicator configured to change according to a movement of the movable member, the at least one indicator being visually recognizable.

### BRIEF DESCRIPTION OF THE DRAWINGS

FIG. 1 is a partial perspective view showing an endoscope in a first embodiment.

FIG. 2 is a top view showing an operation portion of the endoscope in FIG. 1, as viewed from the direction of II in FIG. 1, together with a proximal end side in a longitudinal axis direction of the insertion portion.

FIG. 3 shows the operation portion and the insertion portion in FIG. 2, as viewed from the direction of III in FIG. 2, with only a movable member and an operation lever being shown in a cross section.

FIG. 4 shows a state where an operation lever in FIG. 2 is tilted in a down direction, together with the movable member and the operation portion.

FIG. 5 shows a state where the operation lever in FIG. 4 is tilted in the down direction, as viewed from the direction of V in FIG. 4, with only the movable member and the operation lever being shown in a cross section.

FIG. 6 is a top view showing a modified example of an indicator in FIG. 2, together with the operation portion and the proximal end side in the longitudinal axis direction of the insertion portion.

FIG. 7 shows the operation portion and the insertion portion in FIG. 6, as viewed from the direction of VII in FIG. 6, with only the movable member and the operation lever being shown in a cross section.

FIG. 8 is a top view showing a modified example of an indicator on a finger contact member in FIG. 6, together with the operation portion and the proximal end side in the longitudinal axis direction of the insertion portion.

FIG. 9 shows the operation portion and the insertion portion in FIG. 8, as viewed from the direction of IX in FIG. 8, with only the movable member and the operation lever being shown in a cross section.

FIG. 10 is a top view showing another modified example of the indicator in FIG. 2, together with the operation portion and the proximal end side in the longitudinal axis direction of the insertion portion.

FIG. 11 shows the operation portion and the insertion portion in FIG. 10, as viewed from the direction of XI in FIG. 10.

FIG. 12 is a top view showing a modified example of the indicator in FIG. 10, together with the operation portion and the proximal end side in the longitudinal axis direction of the insertion portion.

FIG. 13 shows the operation portion and the insertion portion in FIG. 12, as viewed from the direction of XIII in FIG. 12.

FIG. 14 is a partial cross-sectional view showing an operation lever, a movable member, and an elastic member in an operation portion of an endoscope in a second embodiment.

FIG. 15 is a top view of the movable member in FIG. 14, as viewed from the direction of XV in FIG. 14.

FIG. 16 is a top view of an indicator in FIG. 14, as viewed from the direction of XVI in FIG. 14.

FIG. 17 is a partial cross-sectional view showing a state where the operation lever in FIG. 14 is tilted.

FIG. 18 is a top view of an indicator in FIG. 17, as viewed from the direction of XVIII in FIG. 17.

FIG. 19 is a top view showing a modified example of the movable member in FIG. 14, together with the operation lever.

FIG. 20 is a top view showing a state where an indicator has moved in accordance with a movement of the movable member in FIG. 19.

FIG. 21 is a top view showing a modified example in which a planar shape of a light-transmitting portion formed in the movable member in FIG. 14 is a rectangular shape.

FIG. 22 is a partial cross-sectional view showing a modified example in which the indicator in FIG. 14 is configured of a spherical member fitted to the operation portion.

### DETAILED DESCRIPTION OF THE PREFERRED EMBODIMENT(S)

Hereinafter, embodiments of the present invention will be described with reference to drawings. In the embodiments to be described below, description will be made by taking a case where an insertion instrument is an endoscope, as an example.

### First Embodiment

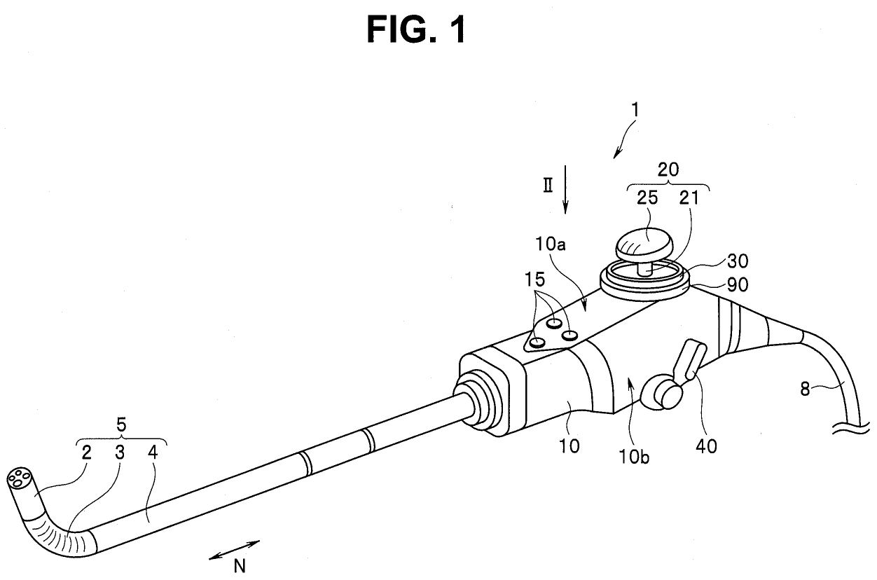

FIG. 1 is a partial perspective view showing an endoscope in the present embodiment.

As shown in FIG. 1, an endoscope 1 includes an insertion portion 5, an operation portion 10, and a universal cord 8. The insertion portion 5 is elongated along a longitudinal axis direction N and configured to be inserted into a subject. The operation portion 10 is provided continuously with a proximal end side in the longitudinal axis direction N of the insertion portion 5. The universal cord 8 is extended from the operation portion 10.

The insertion portion 5 includes a distal end portion 2, a bending portion 3, and a rigid tube 4 that are connected in this order from a distal end side in the longitudinal axis direction N. The bending portion 3 is bendable in any of predetermined directions. The rigid tube 4 is elongated along the longitudinal axis direction N. The predetermined directions include, for example, up, down, left, and right directions, and combined directions (diagonal directions, such as an upper left direction and the like) of two directions, i.e., an up-down direction and a left-right direction.

The endoscope 1 in the present embodiment is configured of a rigid endoscope, for example, a laparoscope, which includes a rigid tube 4 at the insertion portion 5. Note that the bending portion 3 may be configured to actively bend only in two directions, i.e., either the up-down direction or the left-right direction.

The distal end portion 2 includes, inside thereof, an image pickup unit, an illumination unit (neither is shown), and the like. The image pickup unit is configured to observe an inside of a subject and pick up an image of the inside the subject. The illumination unit is configured to supply illumination light to the inside of the subject.

The bending portion 3 includes, inside thereof, a plurality of bending pieces coupled to each other along the longitudinal axis direction N. In addition, in the bending portion 3, distal ends in the longitudinal axis direction N of four wires, not shown, are connected to the bending piece, which is located on the distal-most side in the longitudinal axis direction N, among the plurality of bending pieces, such that the distal ends are positioned at intervals of 90 degrees in a circumferential direction of the bending piece (at rotationally symmetrical positions).

Therefore, the bending portion 3 is configured to bend actively and selectively in a desired direction by any one of the four wires or two of the four wires being selectively pulled by a bending operation device, not shown, provided to the operation portion 10.

The above-described wires, a signal cable extended from the image pickup unit, a light guide configured to supply illumination light to the illumination unit, and the like are inserted through the rigid tube 4.

The operation portion 10 includes a known bending operation device configured to cause the bending portion 3 to bend in any of the above-described predetermined directions.

The bending operation device includes one operation lever 20, a holding lever 40, a movable member 30, and an indicator 35. The operation lever 20 is tilted from a neutral position at which the bending portion 3 is in a non-bending state, to thereby cause the bending portion 3 to bend in any of the above-described predetermined directions.

The holding lever 40 is configured to fix a tilting angle of the operation lever 20, that is, fix a bending state of the bending portion 3.

The operation lever 20 is provided so as to stand up on a top surface 10a in FIG. 1. The top surface 10a is one surface of the operation portion 10.

The operation lever 20 includes a main part constituted of a shaft 21 and a finger contact member 25. The shaft 21 is extended from the inside of the operation portion 10 so as to stand up from the top surface 10a. The finger contact member 25 is provided at an end of the shaft 21.

The movable member 30, which has a substantially circular shape in a planar view from the finger contact member 25 side, is provided around the shaft 21. In the present embodiment, the movable member 30 is configured of an elastic member made of rubber.

The movable member 30 is arranged between an outer circumference of the shaft 21 and an inner circumference of a ring-shaped cover member 90 provided on the top surface 10a of the operation portion 10. The movable member 30 has a function as a watertight member configured to prevent liquid from entering the inside of the operation portion 10 from a hole, not shown, which is formed on the top surface 10a and configured to allow the shaft 21 to pass through.

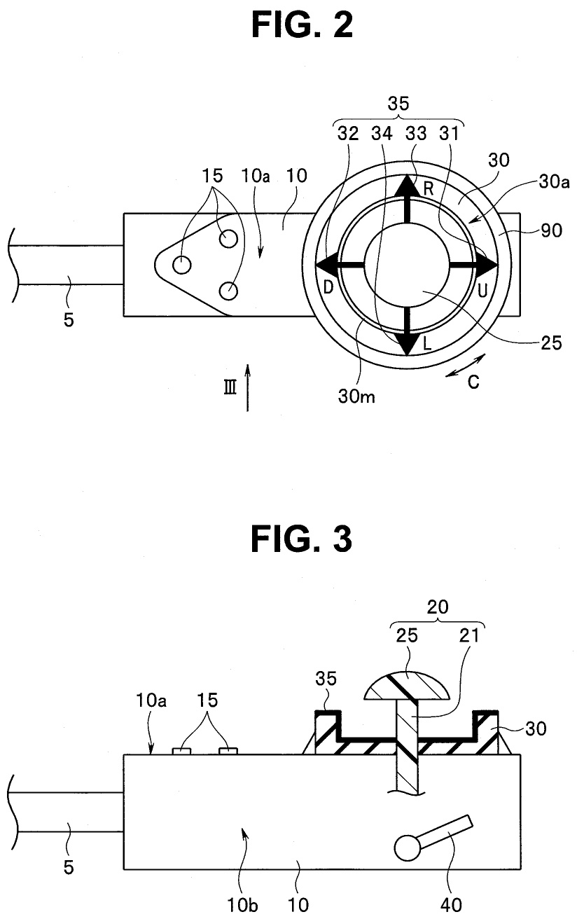

The movable member 30 is configured of the elastic member, and includes a ring-shaped deformation portion 30m (see FIG. 2), to be described later, formed on a surface 30a so as to be located on the finger contact member 25 side. Such a configuration allows the movable member 30 to be deformable in accordance with tilting of the operation lever 20. In other words, the movable member 30 moves according to the tilting of the operation lever 20. The movement of the movable member 30 can be visually recognized by an operator.

In addition, the holding lever 40 is provided on a side surface 10b in FIG. 1. The side surface 10b is continuous with the top surface 10a of the operation portion 10.

When the operator takes the hand off from the finger contact member 25 of the operation lever 20, the operation lever 20 returns to the neutral position, to thereby bring the bending portion 3 into the non-bending state. However, in the case where the hold function is used, the bending state of the bending portion 3 is held even if the operator takes the hand off from the finger contact member 25. Therefore, the state of the bending portion 3 cannot be identified based only on whether or not the tilting operation of the operation lever 20 is performed.

Furthermore, in the case where the operator brings the operation lever back to the neutral position, since the neutral position is judged by the sense of the operator, there is a possibility that the operation lever has not returned to the precise neutral position at which the bending portion 3 is in the non-bending state.

In addition, also in the case where the hold function is not used, depending on the shape or the rigidity of the above-described watertight member, there is a possibility that the operation lever 20 has not returned to the precise neutral position even if the operator takes the hand off from the finger contact member 25.

Since the detailed configuration related to the pulling operation of the four wires by using the operation lever 20 in the bending operation device is known, the description thereof will be omitted. The configuration for fixing the tilting angle of the operation lever 20 by using the holding lever 40 is also known. Therefore, the detailed description of the configuration is also omitted.

The operation portion 10 includes, on the top surface 10a thereof, a button switch 15 to which a given function is assigned from among various functions related to the endoscope 1. Note that the button switch 15 may include one or a plurality of buttons.

Furthermore, the above-described wires, signal cable, light guide, etc., are inserted through the operation portion 10.

The universal cord 8 is extended from the operation portion 10. The universal cord 8 includes, at an extension end thereof, an endoscope connector, not shown.

The above-described signal cable, light guide, etc., are inserted also through the universal cord 8 and the endoscope connector.

The endoscope connector is connected to a light source apparatus, a video processor, and the like, to thereby allow the endoscope 1 to be connected to external apparatuses. An endoscopic image picked up by the endoscope 1 is displayed on a monitor, not shown, of an external apparatus.

Next, with reference to FIGS. 2 to 5, description will be made on a configuration for enabling the operator to visually recognize the above-described neutral position of the operation lever 20.

FIG. 2 is a top view showing the operation portion of the endoscope in FIG. 1, as viewed from the direction of II in FIG. 1, together with the proximal end side in the longitudinal axis direction of the insertion portion. FIG. 3 shows the operation portion and the insertion portion in FIG. 2, as viewed from the direction of III in FIG. 2, with only the movable member and the operation lever being shown in a cross section.

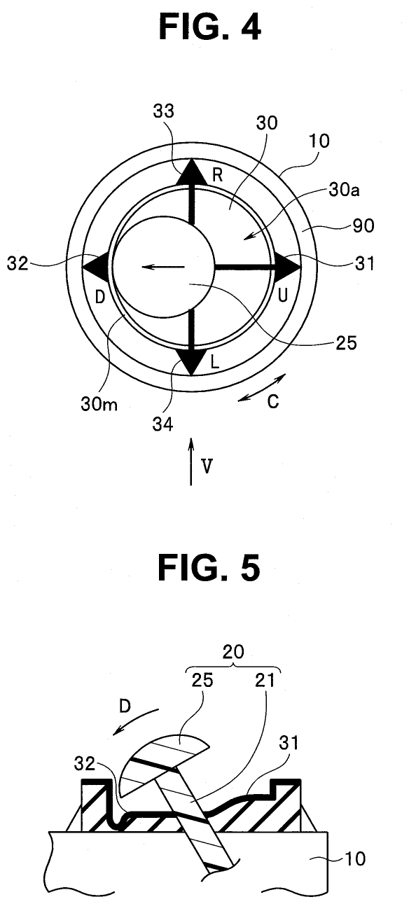

FIG. 4 shows a state where the operation lever in FIG. 2 is tilted in the down direction, together with the movable member and the operation portion. FIG. 5 shows the state where the operation lever in FIG. 4 is tilted in the down direction, as viewed from the direction of V in FIG. 4, with only the movable member and the operation lever being shown in a cross section.

As shown in FIGS. 2 and 3, the indicator 35 is provided on the surface 30a of the movable member 30 serving also as the watertight member. Specifically, the indicator 35 includes a main part configured by including a mark 31, a mark 32, a mark 33, and a mark 34. The marks 31, 32, 33, and 34 indicate an up (U) tilting direction of the operation lever 20, a down (D) tilting direction of the operation lever 20, a right (R) tilting direction of the operation lever 20, and a left (L) tilting direction of the operation lever 20, respectively.

As shown in FIG. 2, the four marks 31 to 34 are formed on the surface 30a of the movable member 30 at rotationally symmetric positions so as to surround the periphery of the operation lever 20, at intervals of substantially 90 degrees in a circumferential direction C.

Furthermore, each of the marks 31 to 34 has a predetermined shape, a predetermined pattern, or a predetermined color. Note that, in the present embodiment, the marks 31 to 34 are described by using an example in which the marks are formed in arrow shapes respectively extending from the shaft 21 of the operation lever 20 toward the cover member 90.

Each of the marks 31 to 34 changes in shape according to the movement of the movable member 30 in accordance with the tilting of the operation lever 20.

As shown in FIG. 4, for example, when the operation lever 20 is tilted in the down direction from the neutral position shown in FIGS. 2 and 3, in the planar view from the finger contact member 25 side, the mark 32 changes in shape such that the visual recognition range by the operator becomes small, and the mark 31 changes in shape such that the visual recognition range by the operator becomes large, compared with the marks 31 and 32 shown in FIG. 2. In other words, the shapes of the marks change according to the movement of the movable member 30.

In addition, as shown in FIG. 5, due to the deformation of the deformation portion 30m, the mark 32 changes in shape so as to create a recess and the mark 31 changes in shape so as to create a bulge and the like, compared with the marks 31 and 32 shown in FIG. 3.

These changes in the shapes of the marks 32 and 31 are not hidden by the finger contact member 25 and are visually recognizable, even if the movable member 30 moves.

Accordingly, the operator can surely visually recognize the changes in the shapes of the marks 32 and 31, and easily visually recognize that the operation lever 20 is tilted precisely in the down direction.

Although not shown, also in the case where the operation lever 20 is tilted in a combined direction (diagonal direction) of the up-down direction and the left-right direction, the shapes of the marks 31 to 34 change according to the movement of the movable member 30, similarly as described above, and the changes are visually recognizable by the operator.

Since other configurations of the endoscope 1 is known, the descriptions thereof will be omitted.

Thus, in the present embodiment, the movable member 30 provided around the shaft 21 of the operation lever 20 moves according to the tilting of the operation lever 20, and the movement of the movable member 30 is visually recognizable by the operator.

In addition, the indicator 35 provided on the surface 30a of the movable member 30 changes in shape according to the movement of the movable member 30, and the change in the shape of the indicator 35 is visually recognizable. In other words, based on the shapes of the four marks 31 to 34 disposed on the movable member 30, the movement according to the tilting of the operation lever 20 (tilting state; tilting amount and tilting direction) can be visually recognized.

With such a configuration, when the operator tilts the operation lever 20 and thereafter releases the finger from the finger contact member 25 to bring the operation lever 20 back to the neutral position shown in FIGS. 2 and 3, or when the operator tilts the operation lever 20 and fixes the tilting angle of the operation lever 20 by using the holding lever 40, and thereafter operates the finger contact member 25 to bring the operation lever 20 back to the neutral position as shown in FIGS. 2 and 3, if the operator visually recognizes that the movable member 30 is not moved and none of the marks 31 to 34 changes in shape by the movement of the movable member 30, the operator can quantitatively and easily visually recognize that the operation lever 20 has returned to the neutral position precisely, that is, the bending portion 3 is in the non-bending state, without depending on the sense of the operator.

As described above, the present embodiment can provide the endoscope 1 configured to be capable of enabling the operator to easily visually recognize the precise neutral position of the operation lever 20, at which the bending portion 3 is in the non-bending state.

### Modified Example of First Embodiment

Hereinafter, modified examples of the first embodiment will be described. In the present modified example, the operator recognizes that the operation lever 20 has returned to the neutral position precisely by visually recognizing the shape of the indicator 35.

The modified example is not limited to the above-described configuration, but the operator may recognize that the operation lever 20 has returned to the neutral position precisely by visually recognizing that the movable member 30 is not moved, that is, the shape of the movable member 30 is not changed. Therefore, the movable member 30 itself may serve as the indicator 35.

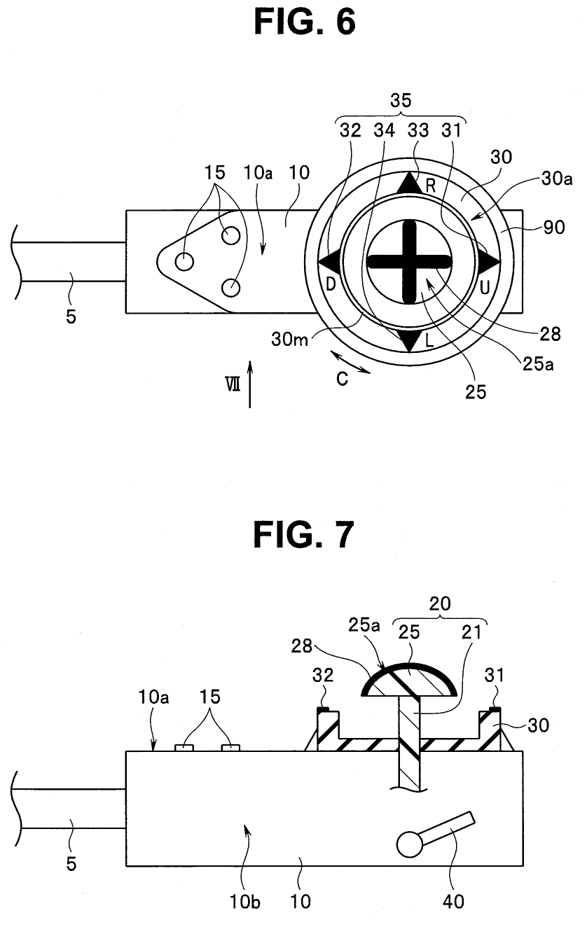

Hereinafter, the modified examples will be described with reference to FIGS. 6 and 7. FIG. 6 is a top view showing the modified example of the indicator in FIG. 2, together with the operation portion and the proximal end side in the longitudinal axis direction of the insertion portion. FIG. 7 shows the operation portion and the insertion portion in FIG. 6, as viewed from the direction of VII in FIG. 6, with only the movable member and the operation lever being shown in a cross section.

As shown in FIG. 6, marks 31 to 34 may be formed only by triangular marks, at the same positions in the circumferential direction C as those shown in FIG. 2. The positions are in a region on the cover member 90 side with respect to the deformation portion 30m on the surface 30a of the movable member 30.

In addition, the finger contact member 25 may include, on a finger contact surface 25a, a mark (second indicator) 28 having a cross shape in a planar view. The ends of the cross shape of the mark 28 are coincident respectively with the marks 31 to 34 in the circumferential direction C, when the operation lever 20 is at the neutral position. Note that other configurations are the same as those in the above-described present embodiment.

With such a configuration, the operator can easily visually recognize the precise neutral position of the operation lever 20 not only by visually recognizing the changes in the shapes of the marks 31 to 34 and the change in the shape of the movable member 30 but also by visually recognizing the coincidence of the marks 31 to 34 with the mark 28 in the circumferential direction C, from the finger contact member 25 side. Other effects are the same as those in the above-described present embodiment.

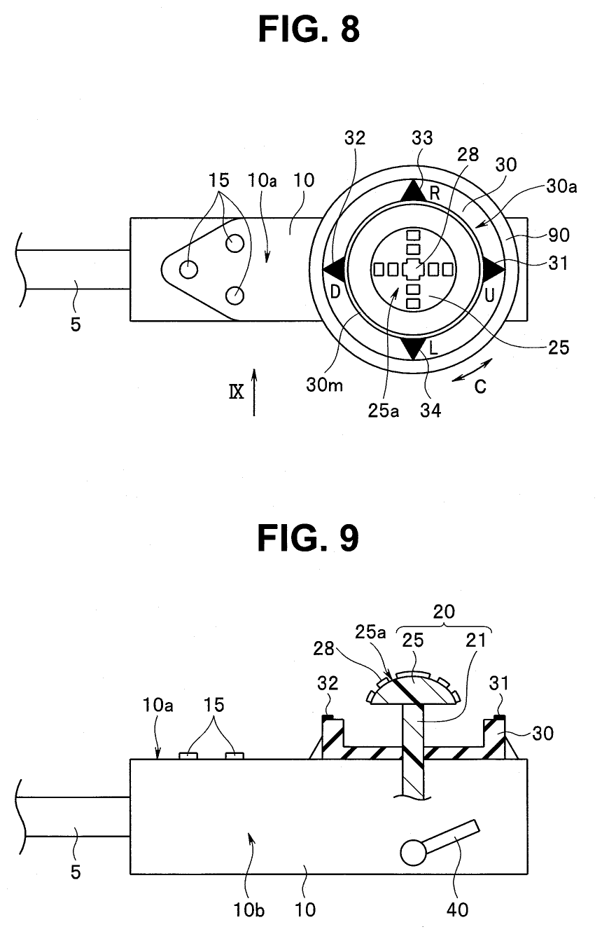

Hereinafter, another modified example will be shown with reference to FIGS. 8 and 9. FIG. 8 is a top view showing the modified example of the indicator of the finger contact member in FIG. 6, together with the operation portion and the proximal end side in the longitudinal axis direction of the insertion portion. FIG. 9 shows the operation portion and the insertion portion in FIG. 8, as viewed from the direction of IX in FIG. 8, with only the movable member and the operation lever being shown in a cross section.

As shown in FIGS. 8 and 9, a cross-shaped mark 28, as an indicator, which is formed on the finger contact member 25, may be a slip stopper constituted of a plurality of protrusions. The mark 28 is not only capable of exhibiting the same effects as those in the configuration shown in FIGS. 6 and 7 but also achieving easy tilting operation by using the finger contact member 25.

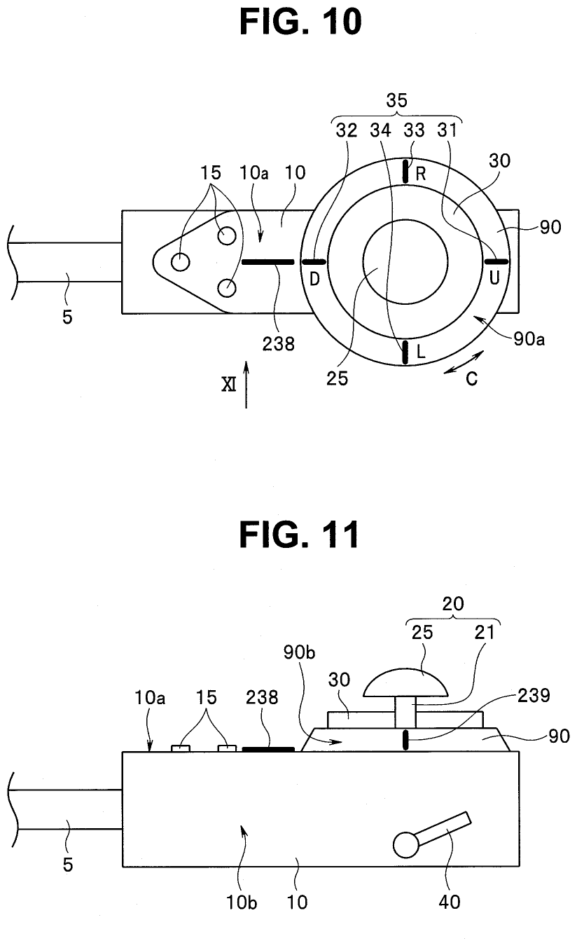

Hereinafter, another modified example will be shown with reference to FIGS. 10 and 11. FIG. 10 is atop view showing another modified example of the indicator in FIG. 2, together with the operation portion and the proximal end side in the longitudinal axis direction of the insertion portion. FIG. 11 shows the operation portion and insertion portion in FIG. 10, as viewed from the direction of XI in FIG. 10.

As shown in FIG. 10, marks 31 to 34 may have bar shapes in the planar view from the finger contact member 25 side. The marks 31 to 34 may be provided not on the movable member 30 but on the surface 90a of the cover member 90 at the same positions in the circumferential direction C as those shown in FIG. 2. In addition, a mark 238, the position of which in the circumferential direction C coincides with that of the mark 32, may be formed also on the top surface 10a of the operation portion 10.

Furthermore, as shown in FIG. 11, a mark 239, as an indicator, may be provided on an outer circumferential side surface 90g of the cover member 90 at the same position in the circumferential direction C as that of the mark 34. Note that other configurations are the same as those in the above-described present embodiment.

With such a configuration, the marks 31 to 34 do not change in shape by the movement of the movable member 30. However, if the movable member 30 does not change in shape, and the operation lever 20 is at the position equidistant from the marks 31 to 34 when viewed from the finger contact member 25 side, the operator can easily visually recognize that the operation lever 20 is precisely at the neutral position.

In addition, when the operator views the side surface 10b side of the operation portion 10, if the shaft 21 is vertical with respect to the mark 238 as the indicator, and the shaft 21 and the mark 239 are aligned on a straight line, the operator can easily visually recognize that the operation lever 20 is precisely at the neutral position. Note that other effects are the same as those in the above-described present embodiment.

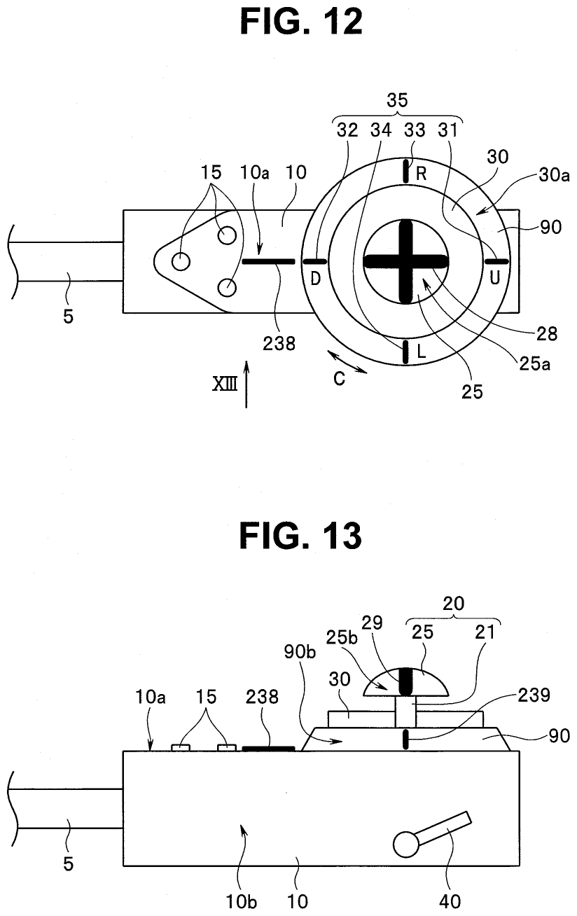

Hereinafter, other modified examples will be shown with reference to FIGS. 12 and 13. FIG. 12 is a top view showing a modified example of the indicator in FIG. 10, together with the operation portion and the proximal end side in the longitudinal axis direction of the insertion portion. FIG. 13 shows the operation portion and the insertion portion in FIG. 12, as viewed from the direction of XIII in FIG. 12.

As shown in FIG. 12, a mark 28 having a cross shape may be formed on the finger contact surface 25a of the finger contact member 25 shown in FIG. 10, in the similar manner as in FIG. 6. Furthermore, as shown in FIG. 13, a mark 29 may be provided on an outer circumferential side surface 25b of the finger contact member 25 at a position corresponding to the position of the mark 239. Note that other configurations are the same as those in FIGS. 10 and 11.

With such a configuration, the marks 31 to 34 do not change in shape by the movement of the movable member 30. However, if the movable member 30 does not change in shape, and the marks 31 to 34 are coincident respectively with the distal ends of the cross shape of the mark 28 in the circumferential direction C, when viewed from the finger contact member 25 side, the operator can easily visually recognize that the operation lever 20 is at the precise neutral position.

In addition, when the operator views the side surface 10b of the operation portion 10, if the mark 29 and the shaft 21 are vertical with respect to the mark 238, and the shaft 21, the mark 239, and the mark 29 are aligned on a straight line, the operator can easily visually recognize that the operation lever 20 is precisely at the neutral position. Note that other effects are the same as those in the above-described present embodiment.

### Second Embodiment

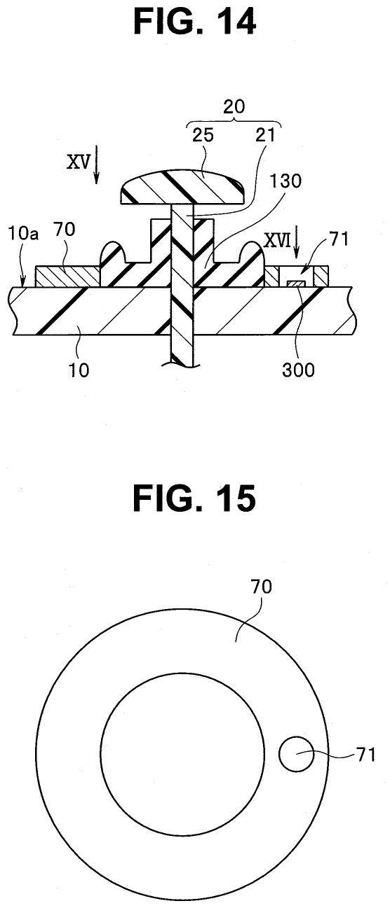

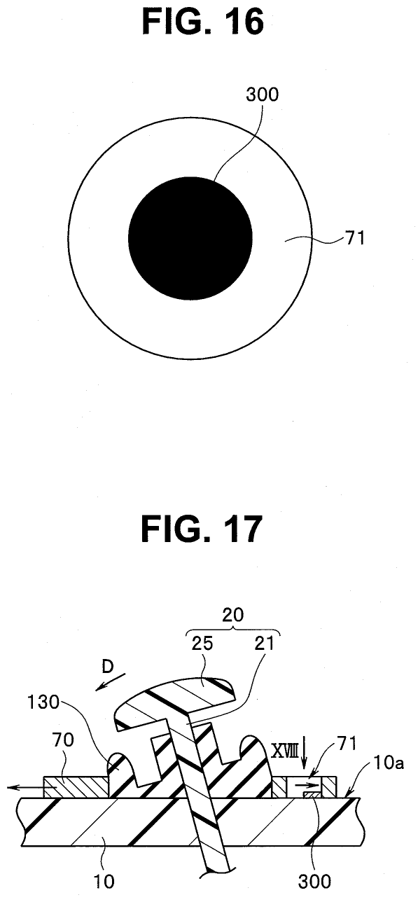

FIG. 14 is a partial cross-sectional view showing an operation lever, a movable member, and an elastic member in an operation portion of an endoscope in the present embodiment. FIG. 15 is a top view of the movable member in FIG. 14, as viewed from the direction of XV in FIG. 14. FIG. 16 is a top view of the indicator in FIG. 14, as viewed from the direction of XVI in FIG. 14.

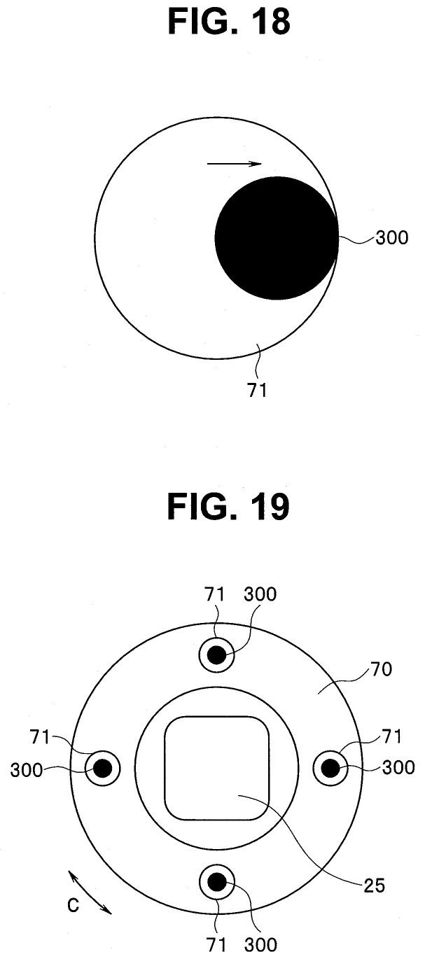

FIG. 17 is a partial cross-sectional view showing a state where the operation lever in FIG. 14 is tilted. FIG. 18 is a top view of the indicator in FIG. 17, as viewed from the direction of XVIII in FIG. 17.

The configuration of the endoscope in the second embodiment is different from the endoscope in the first embodiment in that the movable member is configured of a slide member provided around the elastic member serving as a watertight member.

Therefore, only the different point will be described. The same constituent elements as those in the first embodiment are attached with the same reference signs, and description thereof will be omitted.

As shown in FIG. 14, in the present embodiment, a movable member 70 is provided on the top surface 10a of the operation portion 10 so as to be located around the operation lever 20, and the movable member 70 is configured such that the movement thereof according to the tilting of the operation lever 20 is visually recognizable. The movable member 70 is configured of a slide member having a ring shape in a planar view from the finger contact member 25 side.

Note that the movable member 70 is provided around the elastic member 130 that is provided around the shaft 21. In addition, the elastic member 130 has the same watertight function as that of the movable member 30 shown in the first embodiment.

The movable member 70 slidingly moves on the top surface 10a by being pushed by the elastic member 130, according to the tilting of the operation lever 20, in a direction coincident with the tilting direction of the operation lever 20.

In addition, a mark 300 is provided on the top surface 10a of the operation portion 10 at a position on which the movable member 70 is superposed. The mark 300 is an indicator, the shape of which changes according to the movement of the movable member 70.

Furthermore, in the movable member 70, a light-transmitting portion (light-transmitting region) 71 is formed at a position superposed on the mark 300, as shown in FIGS. 15 and 16. The light-transmitting portion 71 has a circular shape in the planar view from the finger contact member 25 side. The light-transmitting portion 71 is configured such that the mark 300 can be visually recognized therethrough from outside.

Note that the light-transmitting portion 71 is configured of a light-transmitting window or a through hole which is provided in the movable member 70.

In such a configuration, when the operation lever 20 is tilted from the neutral position shown in FIG. 14 in the down direction as shown in FIG. 17, the movable member 70 moves on the top surface 10a to the left side in the drawing by being pushed through the elastic member 130.

As a result, as shown in FIG. 18, the mark 300 moves in the light-transmitting portion 71 from the position shown in FIG. 16 to the right side in FIG. 18. In other words, the indicator changes.

The above-described movement of the mark 300 occurs in the direction opposite to the tilting direction of the operation lever 20 also in the case where the operation lever 20 is tilted in any of the up direction, the left direction, the right direction, and in a combined direction of the two directions, i.e., the up-down direction and the left-right direction.

Therefore, when the operator tilts the operation lever 20 and thereafter releases the finger from the finger contact member 25 to bring the operation lever 20 back to the neutral position shown in FIG. 14, or when the operator tilts the operation lever 20 and fixes the tilting angle of the operation lever 20 by using the holding lever 40, and thereafter operates the finger contact member 25 to bring the operation lever 20 back to the neutral position shown in FIG. 14, if the operator visually recognizes, from the finger contact member 25 side through the light-transmitting portion 71, that the mark 300 is located at the center of the light-transmitting portion 71 as shown in FIG. 16, the operator can quantitatively and easily visually recognize that the operation lever 20 has returned to the neutral position precisely, that is, the bending portion 3 is in the non-bending state.

Other configurations and effects are the same as those in the above-described first embodiment.

### Modified Example of Second Embodiment

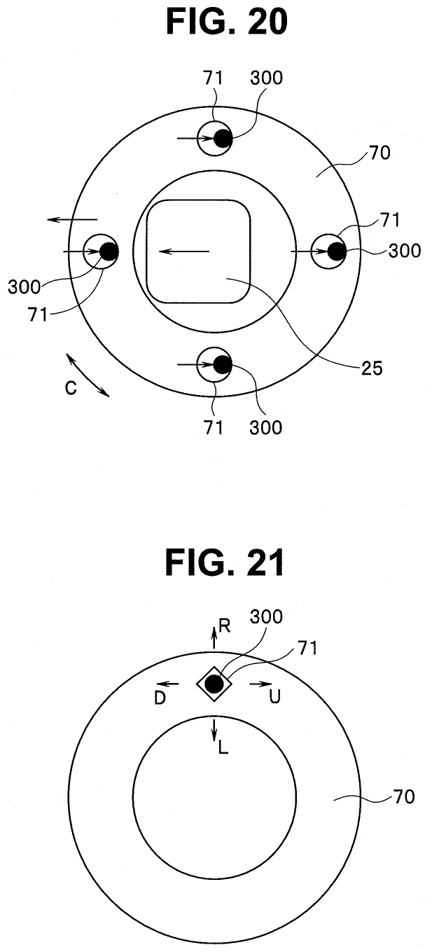

Hereinafter, modified examples of the second embodiment will be described with reference to FIGS. 19 and 20. FIG. 19 is a top view showing a modified example of the movable member in FIG. 14, together with the operation lever. FIG. 20 is a top view showing a state where an indicator has moved in accordance with a movement of the movable member in FIG. 19.

As shown in FIG. 19, a plurality of, for example, four marks 300 may be provided on the top surface 10a at intervals of substantially 90 degrees in the circumferential direction C. The movable member 70 may be provided with four light-transmitting portions 71 through which the four marks 300 can be respectively visually recognized from outside.

With such a configuration, it is easy for the operator to visually recognize the neutral position from a plurality of field-of-view directions by viewing all the four marks 300 respectively from the four light-transmitting portions 71 or any of the four marks 300 from any of the four light-transmitting portions 71.

If the light-transmitting portions 71 are formed respectively at positions corresponding to the up, down, left, and right directions of the operation lever 20, the operator can tilt the operation lever 20 precisely in any of the up, down, left, and right directions by tilting the operation lever 20 toward any of the marks 300 and the light-transmitting portions 71.

Note that other effects are the same as those in the above-described embodiment.

Hereinafter, another modified example will be shown with reference to FIG. 21. FIG. 21 is atop view showing the modified example in which a planar shape of a light-transmitting portion formed in the movable member in FIG. 14 is a rectangular shape.

As shown in FIG. 21, the light-transmitting portion 71 may have a rectangular shape in a planar view from the finger contact member 25 side, such that the corners of the rectangular shape coincide respectively with the tilting directions, i.e., the up, down, left, and right directions of the operation lever 20.

Such a configuration is capable of exhibiting the same effects as those in the above-described present embodiment. In addition, with such a configuration, even if only one light-transmitting portion 71 is provided, the operation lever 20 can be tilted in a precise direction by moving the indicator (mark) toward each of the corners of the light-transmitting portion 71.

Note that, also in the present configuration, the mark 300 and the light-transmitting portion 71 may be constituted of a plurality of marks and a plurality of light-transmitting portions, respectively.

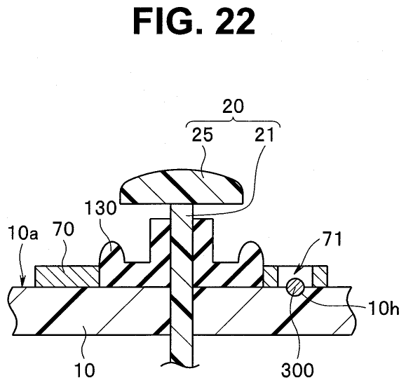

Furthermore, hereinafter another modified example will be shown with reference to FIG. 22. FIG. 22 is a partial cross-sectional view showing a modified example in which the indicator in FIG. 14 is configured of a spherical member fitted to the operation portion.

As shown in FIG. 22, the mark 300 may be configured of the spherical member fitted in a hole 10h formed on the top surface 10a of the operation portion 10.

Such a configuration is capable of exhibiting the same effects as those in the above-described embodiment. In addition, according to such a configuration, when the operator tilts the operation lever 20 and thereafter releases the finger from the finger contact member 25 to bring the operation lever 20 back to the neutral position shown in FIG. 22, or when the operator tilts the operation lever 20 and fixes the tilting angle of the operation lever 20 by using the holding lever 40, and thereafter operates the finger contact member 25, to bring the operation lever 20 back to the neutral position shown in FIG. 22, the light-transmitting portion 71 passes over the mark 300, to thereby generate a click feeling.

Therefore, the operator recognizes the click feeling with the touch of the hand, to thereby be capable of easily recognizing that the operation lever has returned to the neutral position precisely.

Note that, also in the present configuration, the mark 300 and the light-transmitting portion 71 may be constituted of a plurality of marks and a plurality of light-transmitting portions, respectively.

The above-described first embodiment, second embodiment, and modified examples have been described by taking the case where the endoscope 1 is a laparoscope, as an example. However, the present invention is not limited to the example, but may be applicable to a rigid endoscope including another bending portion 3. Furthermore, it is needless to say that the present invention is also applicable to a flexible endoscope having a bending portion.

Note that the above-described first embodiment, second embodiment, and modified examples have been described by taking the case where the insertion instrument is an endoscope, as an example. However, the present invention is not limited to the example, but it is needless to say that the present invention may be applicable also to a treatment instrument, a medical manipulator, and the like.

What is claimed is:

1. An insertion instrument comprising:
an insertion portion configured to be inserted into a subject, the insertion portion including a bending portion configured to be bendable in a predetermined direction;
an operation portion provided on a proximal end side with respect to the insertion portion;
one operation lever provided to the operation portion, the operation lever being configured to tilt, to cause the bending portion to bend in the predetermined direction;
a movable member provided around the operation lever, and configured to move according to tilting of the operation lever; and
at least one indicator configured to change according to a movement of the movable member, the at least one indicator being visually recognizable.

2. The insertion instrument according to claim 1, wherein a tilting amount of the operation lever is visually recognizable based on a change in a shape of the indicator.

3. The insertion instrument according to claim 2, wherein the movable member is an elastic member arranged between the operation lever and the operation portion.

4. The insertion instrument according to claim 3, wherein
the elastic member is a watertight member, and
the indicator has a predetermined shape, a predetermined pattern, or a predetermined color, and is formed on a surface of the watertight member.

5. The insertion instrument according to claim 2, wherein the movable member is a member arranged around the operation lever and configured to slide with respect to the operation portion according to the tilting of the operation lever.

6. The insertion instrument according to claim 5, wherein
the indicator is provided to the operation portion, and
the movable member comprises at least one light-transmitting region through which the indicator is visually recognizable from outside.

7. The insertion instrument according to claim 6, wherein the at least one light-transmitting region is a light-transmitting window or a through hole provided in the movable member.

8. The insertion instrument according to claim 1, wherein the indicator is provided on a surface of the movable member.

9. The insertion instrument according to claim 8, wherein the indicator indicates a tilting direction of the operation lever.

10. The insertion instrument according to claim 9, wherein the indicator includes four marks formed at intervals of substantially 90 degrees in a circumferential direction so as to surround a periphery of the operation lever.

11. The insertion instrument according to claim 4, wherein the predetermined shape is an arrow shape.

12. The insertion instrument according to claim 4, wherein the predetermined shape is a triangular shape.

13. The insertion instrument according to claim 10, wherein the operation lever comprises a finger contact member, and the finger contact member is provided with a second indicator having a cross shape, respective ends of the cross shape being coincident with the four marks when the operation lever is at a neutral position at which the bending portion is in a non-bending state.

14. The insertion instrument according to claim 6, wherein the at least one light-transmitting region comprises a plurality of light-transmitting regions.

15. The insertion instrument according to claim 14, wherein the plurality of light-transmitting regions comprise four light-transmitting regions provided at intervals of substantially 90 degrees in a circumferential direction of the movable member.

16. The insertion instrument according to claim 1, wherein the insertion instrument is an endoscope.
